(19) United States
(12) Patent Application Publication
(10) Pub. No.: US 2026/0272472 A1
(43) Pub. Date: Sep. 17, 2026

(54) RAPIDLY DEPLOYABLE EMERGENCY TOURNIQUET

(71) Applicant: REAcT Medical Inc., Allston, MA (US)

(72) Inventors: Michael FINN-HENRY, Lincoln, MA (US); Jeremy LEVIN, Milwaukee, WI (US); Milad BEHBAHANINIA, Tampa, FL (US)

(21) Appl. No.: 19/670,883

(22) Filed: May 7, 2026

Related U.S. Application Data
(63) Continuation of application No. PCT/US2024/054852, filed on Nov. 7, 2024.
(60) Provisional application No. 63/597,306, filed on Nov. 8, 2023.

(51) Int. Cl. A61B 17/132; A61B 17/00

(52) U.S. Cl. CPC A61B 17/1327; A61B 2017/00407

(57) ABSTRACT

A rapidly deployable tourniquet is provided which includes: a strap configured to be placed around a limb; a tension mechanism configured to store energy; and an activation mechanism in operable communication with the tension mechanism, wherein operation of the activation mechanism is configured to release the energy stored in the tension mechanism to tighten the strap around the limb to a degree that is effective to reduce blood flow through the limb to reduce or eliminate hemorrhage therefrom. In one embodiment, the tension mechanism includes a spool and a pre-tensioned spring operably connected to the spool, wherein operation of the activation mechanism is configured to release the pre-tensioned spring to effectuate rotation of the spool to tighten the strap.

### CROSS-REFERENCE TO RELATED APPLICATIONS

This application is a continuation of International Patent Application No. PCT/US2024/054852, filed Nov. 7, 2024, which claims priority to and benefit of U.S. Provisional Patent Application No. 63/597,306, filed Nov. 8, 2023, each of which is incorporated herein by reference in its entirety.

### BACKGROUND

Tourniquets are frequently used in emergency situations to apply pressure to a hemorrhaging limb to control and arrest bleeding. The pressure applied by the tourniquet should, in most cases, significantly reduce or even stop blood flow through the limb by superseding the arterial pressure of the injured limb. This pressure is critical to the proper functioning of the tourniquet. For example, if the tourniquet does not apply enough pressure to the limb, blood flow may not be sufficiently controlled, i.e. there still may be significant blood loss because of continued blood flow through the limb. Or worse, improper tourniquet application can prevent the outflow of venous blood but allow the inflow of arterial blood, leading to congestion of venous blood within the injured limb, which may worsen bleeding.

Existing tourniquets rely on manual tightening to apply this pressure; however, it may be difficult to obtain and/or retain the requisite pressure using these tightening methods. That is, the user may believe that they have sufficiently tightened the tourniquet, when in reality, the tightness of the tourniquet may not be inhibiting blood flow to the degree necessary to arrest ongoing arterial hemorrhage. Moreover, existing tourniquets do not track time of application in an automated manner and instead require users to manually record tourniquet application time. Prolonged application can lead to muscle damage, vessel thrombosis, or even limb loss, making tourniquet use time an important measure to be known.

It therefore would be desirable to provide an improved tourniquet that 1) facilitates reliable tightening to apply the appropriate amount of pressure to a limb, to mitigate or prevent blood loss, and 2) tracks tourniquet application time to mitigate unnecessarily long use time.

### SUMMARY

In one aspect, a rapidly deployable tourniquet is provided which includes: a strap configured to be placed around a limb; a tension mechanism configured to store energy; and an activation mechanism in operable communication with the tension mechanism, wherein operation of the activation mechanism is configured to release the energy stored in the tension mechanism to tighten the strap around the limb to a degree that is effective to reduce blood flow through the limb to reduce or eliminate hemorrhage therefrom. In one embodiment, the tension mechanism comprises: a spool; and a pre-tensioned spring operably connected to the spool, wherein operation of the activation mechanism is configured to release the pre-tensioned spring to effectuate rotation of the spool to tighten the strap.

In another aspect, a method is provided for reducing blood flow through a patient's limb to reduce or eliminate hemorrhage therefrom, wherein the method includes: placing a strap of a rapidly deployable tourniquet around the limb of the patient; and operating an activation mechanism to release energy stored in a tension mechanism effective to tighten the strap around the limb to a degree that is effective to reduce or eliminate hemorrhaging from the limb. In some embodiments, the method further includes pre-tensioning the strap to reduce slack in the strap after placing the strap and before operating the activation mechanism. In one embodiment, operating the activation mechanism releases a pre-tensioned spring to tighten the strap.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments, Elements and/or components are not necessarily drawn to scale.

FIG. 1A is a perspective view of a rapidly deployable emergency tourniquet, according to one or more embodiments of the present disclosure.

FIG. 1B is a side view of the rapidly deployable emergency tourniquet of FIG. 1A, according to one or more embodiments of the present disclosure.

FIG. 2A is a partial cutaway, perspective view of a tensioning mechanism of the rapidly deployable emergency tourniquet of FIG. 1, according to one or more embodiments of the present disclosure.

FIG. 2B is a partial cross-sectional view of a portion of the tensioning mechanism of FIG. 2A. according to one or more embodiments of the present disclosure.

FIG. 3 is a cross-sectional view of a ratchet mechanism of the spring assembly of the tensioning mechanism show n in FIG. 2, according to one or more embodiments of the present disclosure.

FIG. 4A is a side cross-sectional view of a rapidly deployable emergency tourniquet with a safety mechanism in a first state preventing activation, according to one or more embodiments of the present disclosure.

FIG. 4B is another cross-sectional view of the safety mechanism show n in FIG. 4A, according to one or more embodiments of the present disclosure.

FIG. 5A is a side cross-sectional view of the rapidly deployable emergency tourniquet shown in FIG. 4A with the safety mechanism in a second state permitting activation, according to one or more embodiments of the present disclosure.

FIG. 5B is another cross-sectional view of the safety' mechanism shown in FIG. 5A, according to one or more embodiments of the present disclosure.

FIG. 6A is a cross-sectional view of a safety mechanism of a rapidly deployable emergency tourniquet in a first state preventing activation of the activation mechanism, according to one or more embodiments of the present disclosure.

FIG. 6B is a cross-sectional view of the safety mechanism shown in FIG. 6A in a second state permitting operation of the activation mechanism, according to one or more embodiments of the present disclosure.

### DETAILED DESCRIPTION

A rapidly deployable tourniquet has been developed to more quickly and precisely control extremity' hemorrhage as compared to conventional tourniquets. The presently disclosed tourniquet device advantageously provides that when you press an activation button on the tourniquet device, it releases stored energy' to rapidly tighten a strap around the patient's limb sufficiently to stop blood flow. In contrast, conventional blood pressure cuffs and manually tightened tourniquets rely on pumps and/or energy input from the operator of those devices; they do not have the needed energy already stored and ready to be released to rapidly tension the strap around the limb.

As used herein, the term “rapidly deployable” means that the tourniquet is able to tighten the strap around a limb to an effective pressure in less than 30 seconds, more preferably less than 10 seconds. Generally, this means that the tourniquet can apply a pressure in the range of 200-300 mm Hg to the limb in this time frame, which is effective to reduce blood flow through the limb to reduce or eliminate hemorrhage therefrom.

The rapidly\hat{ }deployable tourniquet generally includes a strap to be placed around a limb (i.e., an arm or a leg of a patient), a tension mechanism, and an activation mechanism, where operation of the activation mechanism is effective to tighten the strap around the limb to a degree that is effective to reduce blood flow through the limb to mitigate negative effects of hemorrhage from said limb. The activation mechanism may be an activation button, which may be depressed to initiate operation of the activation mechanism. In some other embodiments, the activation mechanism may include a dial, a lever, a pull cord, or a sensor. The tension mechanism may be configured to store energy, such as mechanical potential energy'. The tension mechanism may store and release the energy' in non-electrical forms, as it may be desirable that the tourniquet be actuable without the need for a battery, electrical wires, etc.

Use of the rapidly deployable tourniquet may include the following steps: (1) the user places the strap around a limb of a patient in need of control of hemorrhaging from the limb; (2) the user reduces slack in the strap; (3) the user operates the activation mechanism; (4) optionally a timer is started; and (5) the strap is tensioned around the limb. The energy for tensioning the strap may flow from (i) a user applying energy into an activation mechanism (e.g., pressing an activation button), which causes (ii) the rapid release of substantially more energy stored in the tension mechanism, which is used directly to tension the strap.

In some particular embodiments, the tensioning mechanism includes a spool and a pre-tensioned spring disposed within the spool, where depression of the activation button releases the pre-tensioned spring to rotate the spool and tighten the strap.

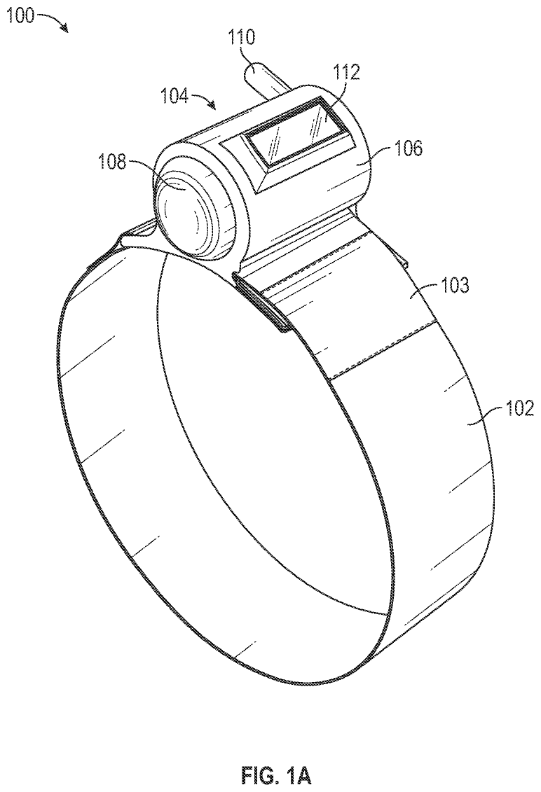

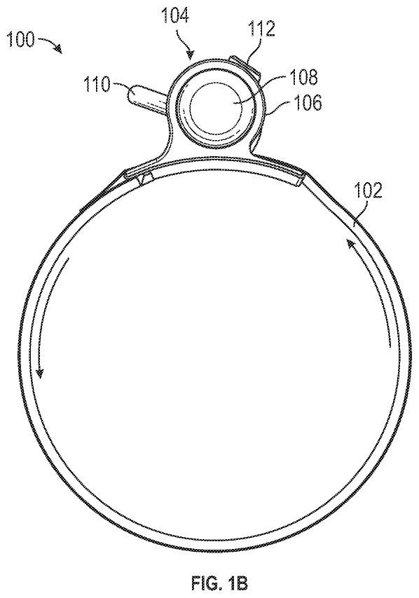

A rapidly deployable tourniquet according to an exemplary' embodiment is illustrated in FIGS. 1A-1B. The rapidly deployable tourniquet 100 includes a strap 102 and a tensioning mechanism 104, which is operationally attached to the strap 102. The strap 102, which may be formed of any suitable flexible material such as nylon, fabric, rubber, or the like, forms a loop that is placed around a hemorrhaging limb above (i.e., proximal to the core of the body) the laceration(s) from which the limb is bleeding. Once the strap 102 is appropriately positioned around the limb, the tensioning mechanism 104 is activated to rapidly and precisely tighten the strap 102 around the limb to a level that is effective to reduce or inhibit blood flow through the limb to mitigate hemorrhaging. The tensioning mechanism 104 has energy' stored therein that, when released, is effective to tighten the strap 102.

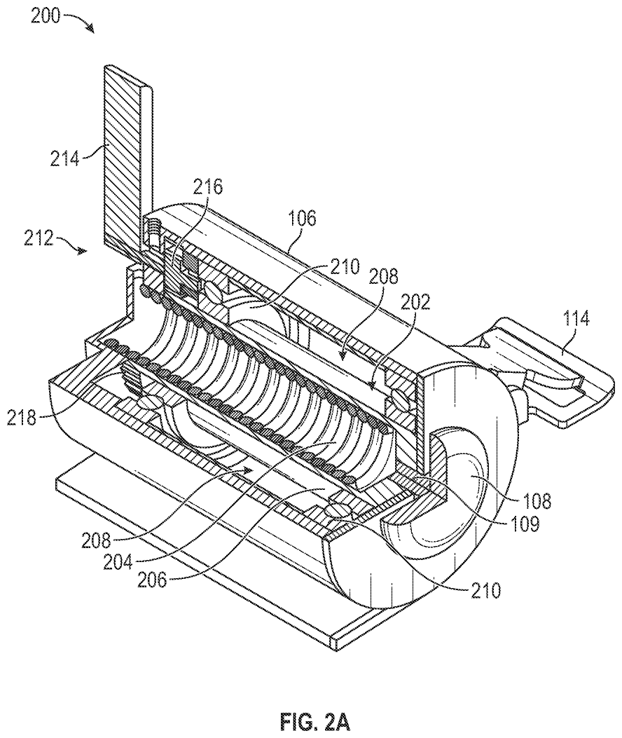

The tensioning mechanism 104 includes a housing 106 which contains the operational components of the tensioning mechanism 104, which is show n in greater detail in FIG. 2. The tensioning mechanism 104 also includes an activation button 108 positioned on an outer surface of the housing 106. Depression of the activation button 108 activates the tensioning mechanism 104 in order to apply tension to the strap 102, thereby tightening the strap 102 around a limb to reduce blood flow and control hemorrhaging.

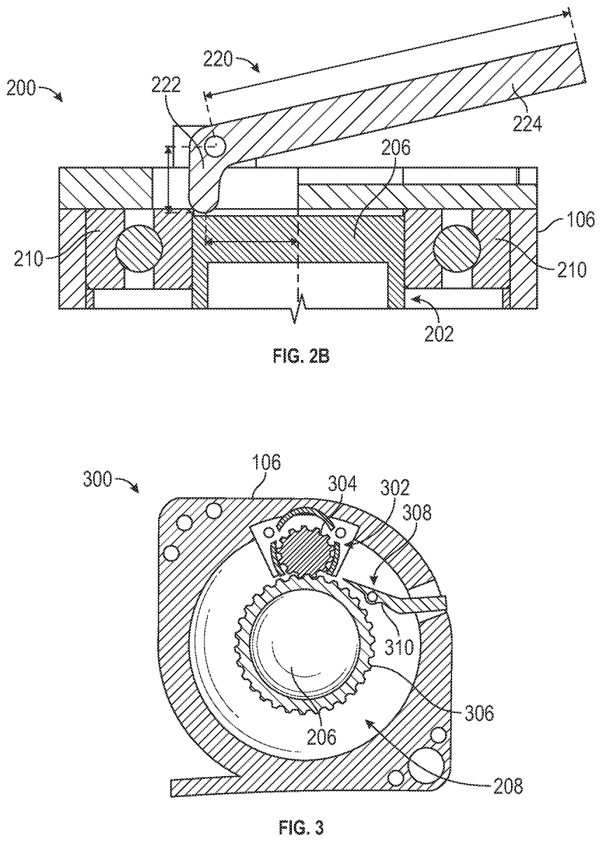

In a preferred embodiment, the tensioning mechanism 104 also includes a manual ratchet mechanism, described in greater detail with respect to FIG. 3, which has an external ratchet arm 110 for further tightening the strap 102. That is, the ratchet arm 110 is connected to components of the manual ratchet mechanism located within the housing 106 of the tensioning mechanism 104. Force is applied to the arm 110 in order to activate the ratchet mechanism to further tighten the strap 102 around the limb if needed, e.g., if the initial tightness of the strap 102 is insufficient to control hemorrhaging from the limb.

The rapidly deployable tourniquet described herein optionally may include a timer configured to measure a period of strap tension being applied to a limb. The timer may be integrated into the actuation mechanism. In one embodiment, actuation of the activation mechanism for triggering strap tensioning may also activate the timer. In some embodiments, the tensioning mechanism 104 also includes a timer 112 disposed on the outside of the housing 106. For example, the timer 112 may be used to indicate to the user when the tourniquet 100 has been applied for a period of time approaching that which could cause damage to the limb from a lack of sufficient blood flow. In this way, the user may know to temporarily loosen the tourniquet. For example, the timer 112 may be activated (i.e., turned on) when the user depresses the activation button 108, thereby eliminating a need for the user to separately record when the tourniquet was placed on the limb. The timer 112 may also have an integrated release mechanism (not show n) that releases tension in the strap 102 upon expiration of the timer. This may be particularly beneficial in situations where the user has applied the tourniquet 100 to their own limb, or where the user may not otherwise be capable of monitoring the timer 112 to determine when the tourniquet 100 should be removed.

The tourniquet 100 also includes a pre-tensioning mechanism that can be used to initially tighten the strap 102 before activating the tensioning mechanism 104. Because the tourniquet 100 may be used with a wide range of limb diameters, the strap 102 must form a loop that is large enough to accommodate most or all limb sizes. However, the excess length in the strap 102 may result in unwanted slack that should be removed before the tourniquet 100 can be used effectively. For example, excess slack in the strap 102 may prevent the tensioning mechanism 104 from properly tightening the strap 102 around the limb, making the tourniquet 100 ineffective. In some instances, the strap 102 may be too long before it is tightened, causing it to move during tightening. This movement may cause the strap 102 to become tangled or caught within the tensioning mechanism 104. In other instances, the tensioning mechanism 104 may not be capable of fully tightening the strap 102 if the strap 102 is too long. The pretensioning mechanism may therefore be used to reduce the length of the strap 102 so that the force applied by the tensioning mechanism 104 is effective to tighten the strap 102 to the necessary degree to control blood flow.

In some embodiments, the pre-tensioning mechanism includes a cam buckle 114 (shown in FIG. 2). In some embodiments, as shown in FIG. 2, the cam buckle 114 may be integrated with or otherwise attached to the housing 106 of the pre-tensioning mechanism. In other embodiments, the cam buckle 114 is positioned at a distance from the housing 106 along the circumference of the strap 102. To use the cam buckle 114 to initially tighten the strap, a free end 103 of the strap 102 is pulled through the cam buckle 114 in a direction away from the pre-tensioning mechanism 104. The end 103 of the strap 102 may continue to be pulled until the desired tightness of the strap 102 is achieved. In some instances, it may be desirable to initially tighten the strap with the cam buckle 114 so that the strap 102 is substantially flush around the limb. In other instances, it may be desirable to simply reduce the amount of slack in the strap 102, without fully tightening the strap 102 to fully contact the limb. The cam buckle 114 may be advantageously configured so that once the strap 102 has been pulled through the buckle 114, the strap 102 may not be loosened unless release of the mechanism 104 is desired to remove the tourniquet, for example, when the patient arrives to surgery to repair the injured limb. That is, the cam buckle 114 utilizes friction or mechanical contact to apply pressure to the strap 102 in a manner that enables motion of the strap 102 in one direction (i.e. the tightening direction) but not the other direction (i.e., the loosening direction).

The tourniquet 100 may be activated, i.e., fully tightened, once the strap 102 has been initially tightened (as needed) by the pre-tensioning mechanism. For example, depressing the activation button 108 may release the energy being stored in the tensioning mechanism 104, which effectively tightens the strap 102 around the limb to the degree necessary to reduce or stop blood flow through the limb. More specifically, activating the tensioning mechanism 104 initiates rotational motion in the strap 102 (as indicated by the arrows in FIG. IB) to “wind up” the excess slack in the strap 102 within the housing 106 of the tensioning mechanism 104. While the rotational motion of FIG. IB is depicted as being counterclockwise, it would be understood that the rotation could be clockwise or counterclockwise, relative to the viewpoint of the user. The extent to which the strap 102 is tightened may depend on the amount of energy stored within the tensioning mechanism 104. That is, the tensioning mechanism 104 may be configured to store an amount of energy that is proportional to the desired tightness of the strap 102.

The tensioning mechanism is shown in greater detail in FIG. 2. The tensioning mechanism 200 includes a spool assembly 202 having a spring 204 disposed within a spool 206. The potential energy' stored in the tensioning mechanism 200 may be stored in the spool assembly 202, particularly the spring 204, where the release of tension in the spring 204 creates the necessary rotational force in the spool assembly 202 to tighten the strap 102 of the tourniquet. That is, the spool assembly 202 is designed to store potential energy via the spring 204, where the release of said energy in the spring 204 is effective to rotate the spool 206 to tighten the strap 102, which is attached to the spool 206. In some embodiments, the strap 102 is integrally formed with the spool 206. In other embodiments, the outer surface of the spool 206 includes at least one attachment point (not shown) to which the strap 102 is attached. The attachment point may be a hole, slit, external attachment fixture, or the like. For example, in embodiments where the attachment point is a hole or slit, the strap 102 may be attached to the hole or slit by a string or secondary attachment means, or the strap 102 may be directly threaded through the hole or slit and secured to itself.

As the spool 206 rotates, the strap 102 may wrap around or wind up on the spool 206. The tensioning mechanism 104 may therefore have a gap 208 between the spool 206 and the inner surface of the housing 106 to accommodate the strap 102 as it is wrapped around the spool 206. The gap 208 may be maintained by at least one spacer 210, or more preferably, a set of spacers 210 positioned proximate to the opposing ends of the spool 206. In some embodiments, the at least one spacer 210 includes a ball bearing and/or a bushing. The size of the spacers 210 may be adjusted so that the gap 208 has an optimal depth for collecting the strap 102. In some embodiments, the gap is maintained by a protrusion from the spool on either side that wraps radially around. Each protrusion may be dimensioned, e.g., 5 mm axially, to reduce contact area and therefore friction between the spool and housing.

In various embodiments, the spring 204 includes one or more torsion springs, torsion bars, constant force springs, or the like. According to a preferred embodiment, the spring 204 is a torsion spring. In the embodiments where the spring 204 includes two or more springs, the two or more springs may be arranged in parallel, in series, or a combination thereof. Springs arranged in parallel may be nested within one another such that the combined spring constant is cumulative (i.e., the total spring constant is the sum of the spring constants for each spring). Springs arranged in series may be attached end to end. According to a preferred embodiment, the spring 204 includes two torsion springs arranged in parallel, where the total combined spring constant is a function of the desired diameter of the strap 102 after it has been tightened. By nesting two or more springs in parallel, the spring force and potential energy stored in the tensioning mechanism 104 can be maximized without having to alter the structure of the other components of the tourniquet 100 engaged with the spring 204. That is, this configuration permits the inclusion of multiple springs 204 within the spool 206 without having to change the length and/or diameter of the spool 206 to accommodate the multiple springs 204. Additionally, providing multiple springs 204 in parallel may more effectively increase the amount of potential energy stored in the tensioning mechanism 104 without increasing the size and/or rigidity of the springs.

For example, in one embodiment, the spring constant is between 8 and 10 N-m, e.g., about 9.3 or 9.4 N-m. The desired spring constant typically is a function of the diameter of the spool. In one embodiment, the goal is to create 370 mm Hg of hoop pressure in the limb which means the force that the spring needs to pull with varies depending on the limb diameter. For a 60 cm diameter limb, about 375 N of tension in the strap would be needed to create this hoop pressure in the limb. This force is then multiplied by the diameter of the spool (40 cm) to get the required spring constant. Because it may be challenging to balance the high spring constant with the high range of motion for the springs in a small package, it may be desirable to nest two or more springs. To achieve a high range of motion, the springs may need to be designed to prevent the wire that the spring is made out of from experiencing too much internal stress, but to get a high spring constant the material of construction may need to be under high material stress. Using two springs instead of one doubles the amount of spring material while keeping with a small form factor, thereby spreading the stress across twice as much material in the same package. In some preferred embodiments, the whole tourniquet assembly is dimensioned to fit with the hand of an average adult.

The spool 206 may have any suitable shape and/or size for housing the spring 204 therein, and may be constructed of any suitable material, such as a metal, plastic, or the like. According to a preferred embodiment, the spool 206 is a cylinder formed of plastic. In a preferred embodiment, the spring 204 is fixedly attached to the spool 206, i.e., the spring 204 is attached to the spool 206 in a manner that prevents motion of the spring 204 at at least one end relative to the spool 206. The spring 204 may be attached to the spool 206 by any suitable means. In some embodiments, the spring 204 may be welded, mechanically fastened, or otherwise attached, to the interior surface of the spool 206. In some embodiments, the ends of the spring 204 may be attached to the spool 206 via holes in the spool 206 sized and shaped to receive the ends of the spring 204. The holes may be positioned along the circumference of the spool 206 or on the flat circular end of the spool 206.

The spool assembly 202 is operationally connected to the activation button 108 by any suitable means. Because of the spring energy being stored in the spool assembly 202, the activation button may include a lever 220. or the like, that contacts the spool 206 to prevent rotational motion thereof while the tourniquet 100 is in its resting or inactivated state. That is, the lever 220 may perpendicularly contact the spool 206, applying a contact force axially or tangentially relative to the rotational direction of the spool 206, with a sufficient amount of force to prevent the spool 206 from rotating. In some embodiments, the lever 220 has a first side 222 in contact with the spool 206 and a longer second side 224 in contact with or attached to the housing 106. The length of the first side 222 of the lever 220 is sufficient to provide enough force to prevent rotational motion of the spool assembly 202, ensuring reliable retention of the tourniquet 100 in the resting state despite the influence of the tensioned spring 204. The longer second side 224 of the lever 220 may be sufficient to create a mechanical advantage to overcome the force preventing rotation of the spool 206 via the contact between the spool 206 and the shorter first side 222 of the lever 220. For this reason, a substantial amount of force may be required to disengage the lever 220 so that the spool 206 can rotate to tighten the strap 102. In some embodiments, the activation button 108 is positioned to that an internal portion 109 of the button 108 moves towards the lever 220 when the button 108 is depressed. For example, when the button 108 is depressed and advanced towards the lever 220, the internal portion 109 of the button 108 may apply a force to the second side 224 of the lever 220, causing it to pivot such that the first side 222 of the lever 220 disengages from the spool 206. With the first side 222 of the lever 220 no longer contacting the spool 206, the spool 206 may be subjected to the force of the spring 204 thereby causing the spool 206 to rotate.

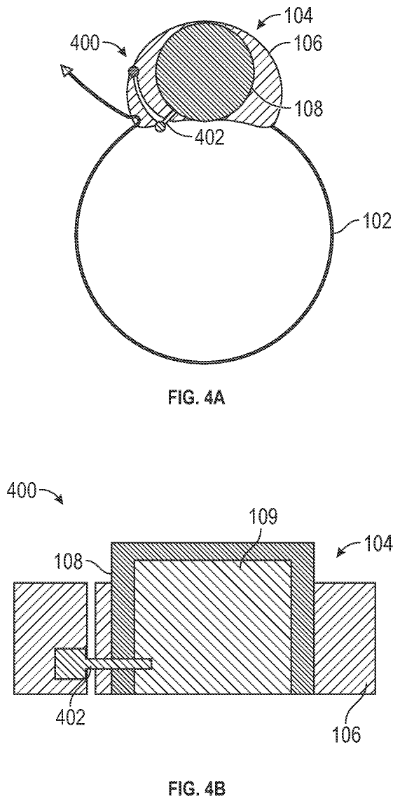

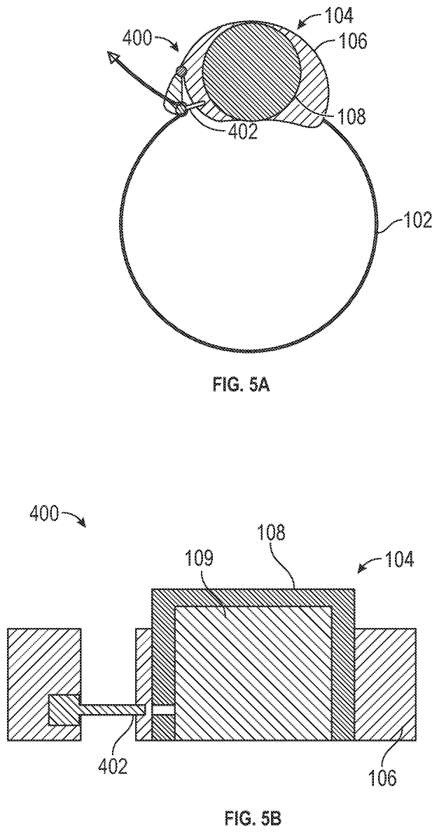

In some embodiments, the tensioning mechanism 104 also includes a safety mechanism for preventing premature or unintended tightening of the tourniquet 100. In some embodiments, as shown in FIGS. 4A-5B, the safety mechanism 400 includes a safety lock 402 that prevents depression of the activation button 108 while the safety lock is in place. For example, the pre-tensioning system may be on a hinge, with a pin preventing the tension activation button from being pressed until some tension is present in the loop. For example, the safety lock 402 may include a pin that contacts the internal portion 109 of the activation button 108 while the safety lock 402 is engaged. The pin 402 may be configured to be released from the internal portion 109 of the activation button 108 when an initial amount of tension is applied to the strap 102 by the pre-tensioner. That is, the safety⁷ lock may also include a spring (not shown) attached to the pin 402 and in contact with the strap 102. When tension is applied to the strap 102 by the pre-tensioner, the spring may pull the pin 402 away from the activation button 108 so that the button 108 can be depressed after the excess slack in the strap 102 is eliminated. The safety lock 402 may therefore allow the button 108 to be depressed only when an initial amount of tension has been applied to the strap 102, i.e. when the tourniquet 100 is in the appropriate condition to be fully tensioned.

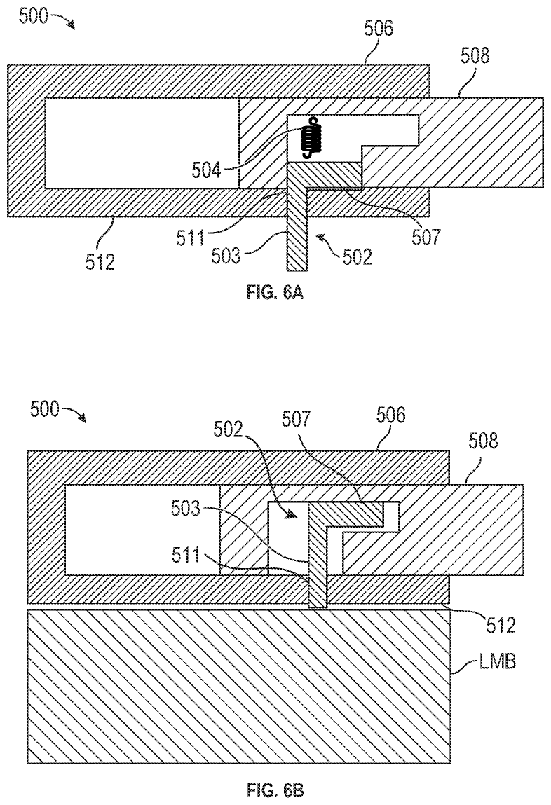

FIGS. 6A-6B shows another embodiment of a safety' mechanism 500. In FIG. 6A, a locking pin 502 is biased into a first position by a spring 504, where in the first position the activation mechanism (or activation button) 508 cannot be displaced to activate the tension mechanism, because a first portion 507 of the locking pin 502 interferes with and prevents translation of the activation mechanism. In the first position, another portion 503 of the locking pin 502 extends through an aperture 511 in an external wall 512 of the housing 506 and protrudes therefrom. In FIG. 6B, the external w all 512 of the housing 506 is brought into contact with a person's limb (LMB), depressing the protruding portion 503 of the locking pin 502 back into the housing, overcoming the bias of the spring. This displaces the locking pin 502 into a second position in which the first portion 507 no longer interferes with the activation mechanism, thereby permitting the activation mechanism to translate and activate and trigger tightening of a strap around the limb.

In some other embodiments, the safety mechanism includes a two-phase engagement process for the activation button 108. For example, in the first phase, depressing the button may release a first lever or latch mechanism engaged with a secondary spring connected to the primary spool mechanism 202, or a separate spool mechanism having a smaller output and a larger range of motion. This spring may initiate an initial rotation of the spool 206. In the second phase, the subsequent or continued depression of the activation button 108 may disengage the primary lever in contact with the spool mechanism 202 to fully release the spring tension stored therein and tighten the tourniquet to its full extent.

In some embodiments, the spool 206 also includes a means for preventing rotation thereof after the tourniquet 100 has been activated, i.e., after the strap 102 has been tightened. For example, the spool 206 may have a slot, divot, or hole configured to interface with a surface that is perpendicular, or nearly perpendicular, to the direction of the rotation of the spool 206. The surface should contact the slot, divot, or hole with sufficient force to prevent rotation of the spool 206 in a direction that would result in loosening of the strap 102, which may help to ensure that the tourniquet 100 is effective in stopping blood loss while in use.

In some embodiments, the tensioning mechanism 200 also includes a manual ratchet system 212 for manually tightening the strap 102 beyond in excess of tension applied to the strap 102 by the tensioning mechanism 104. The manual system 212 is provided only to provide the user with the option to further tighten the strap 102, and otherwise does not interfere with the operation of the tourniquet 100. The manual ratchet system 212 includes a handle 214, a pinion 216, and a plurality of gears 218 disposed around the circumference of the spool 206. The pinion 216, which is indirectly rotated by the handle 214, engages with the gears 218 on the spool 216 to further rotate the spool 206 to provide additional tension (i.e., tightness) to the strap 202. For example, if a patient's limb has a relatively small diameter, the release of the spring 204 tension may not be effective to fully tighten the tourniquet 100 around said patient's limb. The manual ratchet system 212 may therefore be utilized to provide additional tension to the strap 102 so that the tourniquet 100 is tightened around the limb in a manner that is sufficient to restrict blood flow therethrough to prevent excessive blood loss.

The manual ratchet system is illustrated in greater detail in FIG. 3. In some embodiments, the manual ratchet system 300 includes a pinion 302 having a first set of gears 304 configured to engage with a second set of gears 306 disposed around the circumference of the spool 206. An external handle (such as handle 110 shown in FIGS. 1A-1B, or handle 214 shown in FIG. 2) may be directly or indirectly attached to the pinion 302. A user applied force to the handle rotates the pinion 302 and, by way of the mechanical interaction between the first set of gears 304 and second set of gears 306, rotates the spool mechanism 302 in the opposite direction to further tighten the strap 102.

To prevent the manual ratchet 300 from reversing the direction of the spool 306 and unwinding the strap 102 of the tourniquet 100, the manual ratchet 300 may also include a locking mechanism 308. The locking mechanism 308 can be a lever, button, or surface configured to interface with the second set of gears 306. Generally, the locking mechanism 308 should interact with the second set of gears 306 in a manner that permits rotation of the spool 306 in a direction that tightens the strap 102, but prevents rotation of the spool 206 in a direction that loosens the strap 102. The locking mechanism 308 should not otherwise interfere with the rotation of the spool 206. In some embodiments, the locking mechanism includes an arm 310 that interfaces with the gears 306.

### EXEMPLARY EMBODIMENTS

Embodiment 1. A rapidly deployable tourniquet comprising: a strap configured to be placed around a limb; a tension mechanism configured to store energy; and an activation mechanism in operable communication with the tension mechanism, wherein operation of the activation mechanism is configured to release the energy stored in the tension mechanism to tighten the strap around the limb to a degree that is effective to reduce blood flow through the limb to reduce or eliminate hemorrhage therefrom.

Embodiment 2. The rapidly deployable tourniquet of Embodiment 1, wherein the tension mechanism comprises: a spool; and a pre-tensioned spring operably connected to the spool, wherein operation of the activation mechanism is configured to release the pre-tensioned spring to effectuate rotation of the spool to tighten the strap.

Embodiment 3. The rapidly deployable tourniquet of Embodiment 2, wherein the pre-tensioned spring is disposed within the spool.

Embodiment 4. The rapidly deployable tourniquet of Embodiment 2 or 3, wherein the pre-tensioned spring is positioned to drive a gear that engages with the spool.

Embodiment 5. The rapidly deployable tourniquet of any one of Embodiments 2 to 4, further comprising a manual ratchet mechanism configured to manually rotate the spool to tighten the strap around the limb.

Embodiment 6. The rapidly deployable tourniquet of Embodiment 5, wherein the manual ratchet mechanism comprises: a plurality of teeth circumferentially disposed around the spool; a pinion in operational communication with the plurality of teeth; and a handle attached to the pinion and disposed at least partially outside the housing.

Embodiment 7. The rapidly deployable tourniquet of Embodiment 6, wherein rotation of the handle advances the pinion along the plurality of teeth to rotate the spool and tighten the strap around the limb.

Embodiment 8. The rapidly deployable tourniquet of any one of Embodiments 2 to 7, wherein the pre-tension spring comprises a plurality of springs configured to provide tension in the pre-tensioned spring.

Embodiment 9. The rapidly deployable tourniquet of Embodiment 8, wherein the plurality of springs are connected in parallel, such that at least one spring is disposed within at least one other spring.

Embodiment 10. The rapidly deployable tourniquet of Embodiment 8, wherein the plurality of springs are connected in series, such that an end of at least one spring is connected to an end of at least one other spring.

Embodiment 11. The rapidly deployable tourniquet of any one of Embodiments 1 to 10, further comprising a pre-tensioning mechanism configured to reduce slack in the strap prior to release of the tensioning mechanism.

Embodiment 12. The rapidly deployable tourniquet of Embodiment 11, wherein the pre-tensioning mechanism comprises a cam buckle.

Embodiment 13. The rapidly deployable tourniquet of Embodiment 12, wherein the pre-tensioning mechanism is configured to have a free end of the strap pulled through the cam buckle in amount effective to reduce the slack in the strap.

Embodiment 14. The rapidly deployable tourniquet of any one of Embodiments 2 to 13, wherein the tension mechanism further comprises a lever configured to prevent rotation of the spool prior to operation of the activation mechanism.

Embodiment 15. The rapidly deployable tourniquet of Embodiment 14, wherein the lever comprises a first end contacting the spool to prevent the rotation of the spool, and a second end proximate to the activation mechanism.

Embodiment 16. The rapidly deployable tourniquet of Embodiment 15, wherein the activation mechanism is configured to contact the second end of the lever when the activation mechanism is operated, pivoting the lever to remove the first end of the lever from the spool to enable rotation of the spool to tighten the strap around the limb.

Embodiment 17. The rapidly deployable tourniquet of any one of Embodiments 1 to 16, wherein the activation mechanism comprises a safety mechanism configured to prevent unintended operation of the activation mechanism.

Embodiment 18. The rapidly deployable tourniquet of Embodiment 17, wherein the safety mechanism comprises a pin lock configured to block the activation mechanism, and wherein removal of the pin lock enables operation of the activation mechanism.

Embodiment 19. The rapidly deployable tourniquet of Embodiment 17, wherein the safety mechanism comprises a two-stage system for operating the activation mechanism, wherein a first stage comprises operating the activation mechanism to reduce an initial amount of slack in the strap, and wherein a second stage comprises operating the activation mechanism to release the pre-tensioned spring.

Embodiment 20. The rapidly deployable tourniquet of any one of Embodiments 1 to 19, further comprising a timer configured to measure a wear time of the tourniquet, wherein the timer begins upon operation of the activation mechanism.

Embodiment 21. The rapidly deployable tourniquet of Embodiment 20, which is configured to release tension in the strap upon the wear time reaching a predetermined value.

Embodiment 22. A method of reducing blood flow through a patient's limb to reduce or eliminate hemorrhage therefrom, the method comprising: placing a strap of a rapidly deployable tourniquet around the limb of the patient; and operating an activation mechanism to releases energy' stored in a tension mechanism effective to tighten the strap around the limb to a degree that is effective to reduce or eliminate hemorrhaging from the limb.

Embodiment 23. The method of Embodiment 22, wherein operating the activation mechanism releases a pre-tensioned spring to tighten the strap.

Embodiment 24. The method of Embodiment 22 or 23, further comprising pretensioning the strap to reduce slack in the strap after placing the strap and before operating the activation mechanism.

Embodiment 25. The method of any one of Embodiments 22 to 24, further comprising tightening the strap with a manual ratchet mechanism after operating the activation mechanism.

Embodiment 26. The method of any one of Embodiments 22 to 25, wherein the rapidly deployable tourniquet is the rapidly deployable tourniquet of any one of Embodiment 1 to 21.

Modifications and variations of the devices described herein will be obvious to those skilled in the art from the foregoing detailed description. Such modifications and variations are intended to come within the scope of the appended claims.

What is claimed:

1. A rapidly deployable tourniquet comprising:
a strap configured to be placed around a limb;
a tension mechanism configured to store energy; and
an activation mechanism in operable communication, with the tension mechanism,
wherein operation of the activation mechanism is configured to release the energy stored in the tension mechanism to tighten the strap around the limb to a degree that is effective to reduce blood flow through the limb to reduce or eliminate hemorrhage therefrom.

2. The rapidly deployable tourniquet of claim 1, wherein the tension mechanism comprises:
a spool; and
a pre-tensioned spring operably connected to the spool,
wherein operation of the activation mechanism is configured to release the pretensioned spring to effectuate rotation of the spool to tighten the strap.

3. The rapidly deployable tourniquet of claim 2, wherein the pre-tensioned spring is disposed within the spool.

4. The rapidly deployable tourniquet of claim 2, wherein the pre-tensioned spring is positioned to drive a gear that engages with the spool.

5. The rapidly deployable tourniquet of claim 2, further comprising a manual ratchet mechanism configured to manually rotate the spool to tighten the strap around the limb.

6. The rapidly deployable tourniquet of claim 5, wherein the manual ratchet mechanism comprises:
a plurality of teeth circumferentially disposed around the spool;
a pinion in operational communication with the plurality of teeth; and
a handle attached to the pinion and disposed at least partially outside the housing.

7. The rapidly deployable tourniquet of claim 6, wherein rotation of the handle advances the pinion along the plurality of teeth to rotate the spool and tighten the strap around the limb.

8. The rapidly deployable tourniquet of claim 2, wherein the pre-tension spring comprises a plurality of springs configured to provide tension in the pre-tensioned spring.

9. The rapidly deployable tourniquet of claim 8, wherein the plurality of springs are connected in parallel, such that at least one spring is disposed within at least one other spring.

10. The rapidly deployable tourniquet of claim 8, wherein the plurality of springs are connected in series, such that an end of at least one spring is connected to an end of at least one other spring.

11. The rapidly deployable tourniquet of claim 1, further comprising a pre-tensioning mechanism configured to reduce slack in the strap prior to release of the tensioning mechanism.

12. The rapidly deployable tourniquet of claim 11, wherein the pre-tensioning mechanism comprises a cam buckle.

13. The rapidly deployable tourniquet of claim 12, wherein the pre-tensioning mechanism is configured to have a free end of the strap pulled through the cam buckle in amount effective to reduce the slack in the strap.

14. The rapidly deployable tourniquet of claim 2, wherein the tension mechanism further comprises a lever configured to prevent rotation of the spool prior to operation of the activation mechanism.

15. The rapidly deployable tourniquet of claim 14, wherein the lever comprises a first end contacting the spool to prevent the rotation of the spool, and a second end proximate to the activation mechanism.

16. The rapidly deployable tourniquet of claim 15, wherein the activation mechanism is configured to contact the second end of the lever when the activation mechanism is operated, pivoting the lever to remove the first end of the lever from the spool to enable rotation of the spool to tighten the strap around the limb.

17. The rapidly deployable tourniquet of claim 1, wherein the activation mechanism comprises a safety mechanism configured to prevent unintended operation of the activation mechanism.

18. The rapidly deployable tourniquet of claim 17, wherein the safety mechanism comprises a pin lock configured to block the activation mechanism, and wherein removal of the pin lock enables operation of the activation mechanism.

19. The rapidly deployable tourniquet of claim 17, wherein the safety mechanism comprises a two-stage system for operating the activation mechanism, wherein a first stage comprises operating the activation mechanism to reduce an initial amount of slack in the strap. and wherein a second stage comprises operating the activation mechanism to release the pre-tensioned spring.

20. The rapidly deployable tourniquet of claim 1, further comprising a timer configured to measure a wear time of the tourniquet, wherein the timer begins upon operation of the activation mechanism.

21. The rapidly deployable tourniquet of claim 20, which is configured to release tension in the strap upon the wear time reaching a predetermined value.

22. A method of reducing blood flow through a patient's limb to reduce or eliminate hemorrhage therefrom, the method comprising:
placing a strap of a rapidly deployable tourniquet around the limb of the patient; and
operating an activation mechanism to releases energy stored in a tension mechanism effective to tighten the strap around the limb to a degree that is effective to reduce or eliminate hemorrhaging from the limb.

23. The method of claim 22. wherein operating the activation mechanism releases a pre-tensioned spring to tighten the strap.

24. The method of claim 22. further comprising pre-tensioning the strap to reduce slack in the strap after placing the strap and before operating the activation mechanism.

25. The method of claim 22, further comprising tightening the strap with a manual ratchet mechanism after operating the activation mechanism.
